(19) United States
(12) Patent Application Publication
(10) Pub. No.: US 2005/0266105 A1
(43) Pub. Date: Dec. 1, 2005

(54) Compositions comprising natural agents for the treatment of HIV-associated opportunistic infections and complications and methods for preparing and using compositions comprising natural agents

(76) Inventors: Kwame Titus Ashiagbor, Accra (GH); Stephen Ashiagbor, Accra (GH); Anthony K. Wutoh, Upper Marlboro, MD (US); Yaw Foster Kallia, Accra (GH); Rita Delores Wutoh, Upper Marlboro, MD (US); Jeffrey K. Wutoh, Brookville, MD (US); Elnora Aidoo, Accra (GH)

Correspondence Address: ANTHONY K. WUTOH, 17340 Queen Ann Road, Upper Marlboro, MD, 20774, US

(21) Appl. No.: 11/062,769

(22) Filed: Feb. 22, 2005

Related U.S. Application Data
(60) Provisional application No. 60/545,508, filed on Feb. 19, 2004.

(51) Int. Cl. A61K 35/78

(52) U.S. Cl. 424/736; 424/769

(57) ABSTRACT

The present invention pertains to compositions for the treatment of HIV-related opportunistic infections and complications. More specifically, the present invention is directed to a composition comprising Zanthoxylum gillettii and Anogeissus leiocarpus, in combination with citrus juice, or biomass extracts isolated therefrom, and methods of using and manufacturing the same.

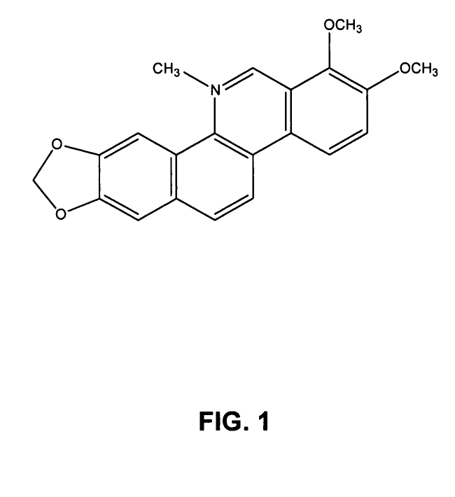

### CROSS REFERENCE TO RELATED APPLICATIONS

The application claims the benefit of U.S. Provisional Patent Application No. 60/545,508, filed Feb. 19, 2004, which is hereby incorporated by reference.

### STATEMENT REGARDING SPONSORED RESEARCH OR DEVELOPMENT

“Not Applicable.”

### REFERENCE TO SEQUENCE LISTING

“Not Applicable.”

### BACKGROUND OF THE INVENTION

1. Field of the Invention

The present invention pertains to compositions for the treatment of HIV-related opportunistic infections and complications. More specifically, the present invention is directed to a composition comprising biomass extracts isolated from Zanthoxylum gillettii and Anogeissus leiocarpus, in combination with citrus juice, and methods of using and manufacturing the same.

2. Description of Related Art

HIV (Human Immunodeficiency Virus) is a retrovirus that ultimately integrates into the host cell genome. The virus attaches to the CD4 molecule on the host cell surface, enters the cell, and begins its replicative cycle which results in destruction of the CD4 T cell. Patients with HIV therefore exhibit diminished proliferation of CD4 T cells.

AIDS (Acquired Immunodeficiency Syndrome) is an immunodeficiency disease that is caused by HIV. AIDS results in immunologic deficit, more specifically, an impairment in cell-mediated immunity resulting from a significant loss of T-cells bearing the CD4 marker. These T cells coordinate a number of critical immunologic functions. The loss of these cells results in the progressive impairment of the immune system and is associated with a deteriorating clinical course. In advanced AIDS, abnormalities of virtually every component of the immune system are evident. Patients frequently succumb to slow, progressive wasting disorders, neurodegeneration, diarrhea, wasting, opportunistic infections and death.

HIV and the resultant disease of AIDS continue to spread rampantly and plague millions of people worldwide. Recent reports suggest that over 42 million people are infected with the HIV virus internationally, with over 3.2 million deaths due to AIDS in 2002 alone. Developing nations in Africa and Asia have been particularly hard hit, with some countries reporting infection rates as high as 30-40% in their adult populations.

The vast majority of the world's infected populations live in countries where diagnostic facilities may be minimal. These developing countries also have little or no financial means of accessing innovative synthetic pharmaceuticals and medications. Moreover, there is a shortage of trained medical professionals and practitioners in the countries most affected by HIV. The financial means and medical structure in many developing nations highlight not only the great need for HIV-related therapies, but also demonstrate the need for affordable alternatives to western medicine.

In some developing parts of the world, where modern medicine and trained personnel are lacking, traditional healers are regarded as an important national health resource. It is estimated that traditional healers are used by 80% of South Africans, where cultural medicine is widely accepted and respected. Herbal medication is the most common therapeutic method used by traditional healers.

Researchers have been exploring new avenues of treating HIV-related symptoms. Recently, there has been much interest in investigating whether natural substances, as opposed to purely synthetic substances, may alleviate opportunistic infections. However, these natural agents are often less active than most HIV-related drugs. Consequently, while herbal remedies have found utility in alleviating certain HIV-related symptoms, the reports are not consistent and the efficacy of the natural agents has been questioned.

There are a number of studies demonstrating that herbs with anti-microbial and/or anti-fungal activity yield the most promising results for treating various viral infections. For example: Tatsadjieu L. N., et al., Fitoterapia (2003) 74(5):469-72 discloses the anti-microbial activity of several herbal extracts, including extracts of Xylopia aethiopica, Monodora myristica, Zanthoxylum xanthoxyloides and Z. leprieurii. Matsu E. N., et al., J. Ethnopharmacol (2003) 87(1):3-41 have shown that the highest anti-microbial activity was found in the methanol extracts of Maytenus senegalensis, Plectranthus barbatus, Zanthoxylum chalybeum, and Zanthoxylum usambarense. Zanthoxylum plants have also shown some antimalarial activity. See, Weenan H., et al., Planta Med (1990) 56(4):371-3. In addition, Vonthron-Senecheau C., et al., J. Ethnopharmacol (2003) 87(2-3):221-25; and Taiwo O., et al., Phytother Res. (1999) 13(8):675-79 have demonstrated that Anogeissus leiocarpus has shown activity against a variety of viruses, including maleria and Staphylococcus aureus.

The specific antimicrobial activity of solvent-fractionated extracts of Anogeissus leiocarpus against Methicillin-Resistant Staphylococcus aureus 595445 and Burkohideria cepacia 92.443 are shown below in Table 1.

TABLE 1

| Extract of Anogeissus | | |
|---|---|---|
| leiocarpus (310 g) | MRSA^{d} | B. cepacia |
| Methanol | 19.5 | 21.5 |
| Chloroform | 12.5 | 0 |
| Ether | 11.5 | 10.5 |
| Butanol | 10.0 | 0 |
| Residue | 19.5 | 20.5 |

*Methanol extracts of Anogeissus leiocarpus (310 g) suspended in water (500 ml) and extracted successively with chloroform (1.5 L × 4), ether (1. L × 4), and butanol (1.5 L × 4) to obtain chloroform soluble fractions.
^{d}Diameter of zone of inhibition in mm. 0 mm refers to no visible zone of inhibition. Diameter of well was 7 mm. See, Taiwo O, et al., Pytotherapy Research, (1999) 13: 675-679.

The antimicrobial activity of aqueous extracts of Anogeissus leiocarpus against various pathogens is demonstrated in Table 2 below.

TABLE 2

| | Anogeissus leiocarpus | ^{d}Diameter |
|---|---|---|
| | Concentration (%, w/v of | of zone of |
| Pathogen | solution applied to wells) | inhibition |
| Streptococcus mutans | 20 | 10.0 |
| NG8 | | |
| Pseudomonas | 20 | 20.5 |
| aeruginosa | | (partial inhibition) |
| Group B Strep A909 | 20 | 20.0 |
| Klebsiella pneumoniae | 20 | 18.5 |
| Salmonella typhimurum | 20 | 12.0 |
| Enterocuccus faecium | 20 | 12.0 |
| #228 | | |

^{d}Diameter of zone of inhibition in mm. 0 mm refers to no visible zone of inhibition. Diameter of well was 7 mm. (Taiwo O., et al., Phytotherapy Research, (1999) 13: 675-679).

The effectiveness of an extract of Zanthoxylum gillettii bark was tested by determining the Minimum Inhibitory concentrations (MIC's) for test organisms against MRSA, in comparison with the antibiotics Norfloxacin and Erythromycin. The results are shown below in Table 3.

TABLE 3

| | Zanthoxylum extract | | |
|---|---|---|---|
| MRSA | (Chelerythrine)* | | |
| Strains | (MIC) - (ug/ml) | Norfloxacin | Erythromycin |
| RN4220 | 8 | 32 | 64 |
| XU212 | 16 | 8 | 4096 |
| 1199-B | 8 | 64 | 2 |
| ATCC | 4 | 16 | 0.25 |
| 25923 | | | |

*Powdered bark (2 Kg) was suspended for one week in 80% methanol, 20% water (10 L). After filtration, the solvent was removed under vacuum. The crude extract was suspended in sulphuric acid (2%) and pH adjusted to 1. (Gibbons, S., et al., Phytotherapy Research, (2003) 17: 274-275).

There are a few reports on small combinations of natural agents that suggest that when used in combination, they can produce effects that appear to alleviate HIV-related symptoms. Wange J., et al., J. Tradit. Chin. Med. (2002) 22(2):93-98 discloses that a Chinese herbal supplement with a combination of herbs called Zhongyan-2 Recipe (ZY-2) shows some alleviation of certain AIDS symptoms. Usha P. R., et al., Drugs RD (2003) 4(2): 103-09 teaches a combination of Indian herbal compounds (Immu-25) that have also been investigated. When administered to patients with HIV, Immu-25, a multi-herbal product, was shown to yield a decrease in the incidence and severity of symptoms including diarrhea, fatigue, anorexia, cough and fever. Other Chinese and Mongolian herbal extracts have been studied for activity against HIV. Several of the more active compounds were identified as alkaloids. (Ma C. M., et al., Phytother Res. (2002) 16(2): 186-89). Other potentially beneficial herbal extracts and compounds that have been investigated for anti-HIV properties include Garcinia kola Heckel (Clusiaceae), Aristolochia ringens Vahl (Aristolochiaceae), Ocimum gratissimum Linn (Lamiaceae), Alstonia boonei DeWild. (Apocynaceae), Cassia podocarpa Guil et Perr. (Caesalpinoideae), Zanthoxylum zanthoxyloides Waterm (Rutaceae), and Heliotropium indicum Linn (Boraginaceae). (Elujoba T., et al., Presented at the FDA Regional Conference on Statergies for Combating the Spread of HIV/AIDS in West Africa at Abuja, Nigeria, Jun. 5-8, 2000).

As previously described, studies have been conducted investigating the effectiveness of individual and multiple herbal components. However, to date, no studies have been conducted using the specific combination of products presented in the present invention in the treatment of HIV disease. Further, no studies have suggested that the combination of these natural agents as disclosed in the present invention can produce additive or supra-additive (synergistic) effects. Nor have studies investigated methods of producing and using the individual components for the treatment of HIV-related opportunistic infections and complications.

Many factors affect the anti-microbial and anti-fungal activity of herbal compounds, including, but not limited to, selecting, harvesting and processing raw herbs and compounds, optimising methods of extraction, standardizing amounts and combinations of extracts, improving yields of extraction, extracting with more potent solvents, and improving the methods of administration.

There is still a need, therefore, for a therapeutic composition comprising natural agents that when combined result in a synergistic effect that is useful in the treatment of HIV-related opportunistic infections and complications. Further there exists a need for a process of using and manufacturing compositions comprising natural agents that when combined result in a synergistic effect that is useful in the treatment of HIV-related opportunistic infections and complications.

### SUMMARY OF THE INVENTION

In none of the studies reviewed is the combined effect of the anti-microbial and anti-fungal activities of biomass extracts isolated from Zanthoxylum gillettii and Anogeissus leiocarpus, in combination with fresh squeezed lime juice, considered in the treatment of HIV associated opportunistic infections and complications.

The present invention provides compositions for the treatment of HIV-related opportunistic infections and complications comprising Zanthoxylum gillettii and Anogeissus leiocarpus, in combination with citrus juice, wherein the anti-microbial and anti-fungal activities of the individual agents are increased relative to the anti-microbial and anti-fungal activities of the individual agents when used alone. As such, the composition of the present invention is effective for the treatment of HIV-related opportunistic infections and complications, including wasting.

The present invention also provides a composition comprising biomass extracts isolated from Zanthoxylum gillettii, Anogeissus leiocarpus, and citrus juice, wherein the biomass extracts contains active agents in the range from 20 μg/ml to 300 μg/ml.

While not wishing to be bound by any one theory, the inventors hypothesize that the compounds comprising the composition of the present invention display anti-microbial and/or anti-fungal activity appear to treat symptoms and infections associated with HIV by operating in the body to decrease viral load, increase CD4 count, increase weight, and decrease the incidence and severity of symptoms including diarrhea, fatigue, anorexia, cough and fever.

The present invention further provides methods for treating HIV-related opportunistic infections and complications by administering to a host inflicted with HIV-related opportunistic infections and complications, such as wasting, a therapeutically effective amount of a composition comprising Zanthoxylum gillettii, Anogeissus leiocarpus, and citrus juice, or biomass extracts isolated therefrom, to a human or mammal.

To achieve the foregoing and in accordance with the purposes of the present invention, as embodied and broadly described herein, the present invention provides compositions comprising Zanthoxylum gillettii and Anogeissus leiocarpus, in combination with citrus juice, or biomass extract isolated therefrom, to form a single therapeutic formulation, wherein each of the natural agents has anti-microbial and anti-fungal activities and wherein the natural agents in the composition interact additively and/or synergistically to enhance their abilities to treat HIV-related opportunistic infections and complications.

To further achieve the foregoing in accordance with the purposes of the present invention, as embodied and broadly described herein, the present invention further comprises methods of extracting the compounds contained within natural agents to achieve increased anti-microbial and anti-fungal activity, resulting in a therapeutically effective result.

Thus, the present invention provides methods for isolating a mixture of biomass extracts from organic compounds comprising subjecting a starting material to liquid extraction with a liquid organic extractant in which said biomass extracts are soluble; recovering said biomass extracts from said extractant whereby said biomass extracts are isolated from said organic compounds contained in said starting material.

The present invention also provides methods for isolating a mixture of biomass extracts from organic compounds comprising subjecting a starting material to methanol thereby producing an extractant; drying said extractant; adding said extractant to water; subjecting said extractant to ether to obtain chloroform soluble fractions; recovering said biomass extract from said extractant whereby said biomass extract is isolated from said organic compounds contained in said starting material.

Prior to this invention, boiling plant products in water to extract potentially active ingredients has produced herbal supplement products for the treatment of HIV-related opportunistic infections and complications. Also prior to this invention, methods of using herbal supplement products for the treatment of HIV-related opportunistic infections and complications have consisted of herbal teas, orally administered solutions, tonics (administered orally), orally administered powders, and topical agents.

Improvements in selecting, harvesting and processing raw herbs and compounds may be obtained by employing the techniques enumerated herein. Further, improvements in the yield may be obtained by employing the techniques enumerated herein including, but not limited to, extraction with non-aqueous solvents, and greater optimization of the temperatures for extraction. Further still, improvements in the amount absorbed into the body can be obtained through administration routes enumerated herein, including, but not limited to, routes based upon the structure of the active compounds, and pharmacokinetic determinants.

To further achieve the foregoing in accordance with the purposes of the present invention, as embodied and broadly described herein, the present invention further comprises products useful for treating HIV-related opportunistic infections and diseases comprising following the aforementioned methods of extracting the compounds contained within natural agents to achieve increased anti-microbial and anti-fungal activity, resulting in a therapeutically effective result.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out herein.

### DETAILED DESCRIPTION OF THE INVENTION

The present invention discloses compositions and methods of treating HIV-related opportunistic infections and complications. In general, the compositions of this invention comprise biomass extracts isolated from Zanthoxylum gillettii and Anogeissus leiocarpus, in combination with citrus juice to produce a therapeutic formulation, wherein each or some of the natural agents possesses anti-microbial and anti-fungal activity.

While not wishing to be bound by any theory, it is believed that the natural agents in the compositions interact additively and/or synergistically to enhance the anti-microbial and anti-fungal activities of the composition. Because of these interactions between the natural agents comprising the present compositions, it was discovered that the concentration of each natural agent in composition of this invention could significantly improve the efficacy of the anti-microbial and anti-fungal activity of the composition. It is the combination of the three natural products that that shows synergistic effect in the range of infections that can be treated, and in the increase of appetite and improvement in weight gain for identified patients.

Methods of using the composition of the present invention and administering to the host a therapeutically effective amount of a composition of this invention are further disclosed. The present invention further provides a method of reducing CD4 T-cell depletion, comprising extracting anti-microbial and anti-fungal agents from a composition of this invention and administering to a host a therapeutically effective amount of a composition of this invention.

The basis of the present invention is the finding that certain plants contain anti-microbial and anti-fungal activities that assist in the treatment of HIV-associated opportunistic infections and complications and in the reduction of CD4 T cell depletion. Further, it was discovered that useful and exploitable levels of these anti-microbial and anti-fungal activities occur in a specific blend comprising biomass extracts isolated from Zanthoxylum gillettii and Anogeissus leiocarpus, in combination with citrus juice to produce a single therapeutic formulation, whereby the active agents work additively and/or synergistically to enhance the anti-microbial and anti-fungal activities of the other agents in the composition. This offers the advantage of allowing therapeutically effective doses of natural agents to be used as compared to the dose of synthetic substances that would be required in order to achieve the same or similar therapeutic effect. Until this invention, it was not known that natural agents extracted from the biomasses of Zanthoxylum gillettii and Anogeissus leiocarpus, in combination with citrus juice, each of which individually possess different anti-microbial and anti-fungal activities, could be combined into one single formulation that would possess the ability to treat HIV-related opportunistic infections and complications and reduce the depletion of CD4 T cells.

As stated above, the compositions of this invention comprise natural agents extracted from the biomasses of Zanthoxylum gillettii and Anogeissus leiocarpus, in combination with citrus juice, combined into a single formulation. In particular, the compositions of this invention are preferably natural agents extracted from the biomasses of Zanthoxylum gillettii and Anogeissus leiocarpus, in combination with fresh squeezed limejuice.

The major chemical constituents that have been identified in the natural agents of the composition of the present invention are shown in Table 4.

TABLE 4

| Botanical Name | Latin Binomials | Common Description | Major Chemical ingredients |
|---|---|---|---|
| Anogeissus | Anogeissus | ATARA, AYIN, | COUMARIN |
| leiocarpus | leiocarpa | KASSANKI, KEDELI, | FLAVELLAGIC ACID, 3-3′-4- |
| | Anogeissus | KEREKETE, KERKETE, | TRIMETHOXY |
| | schimperi | KODYOLI, MARIKE, | 4′-O-BETA-D′-GLUCOSIDE |
| | Conocarpus | MARKE, N′GALAMA, | 3,4,3′-TRI-O- |
| | leiocarpus | NGALAMA, PAKO AYIN, | METHYLFLAVELLAGIC ACID |
| | | PAKO DUDU, SAHAB | |
| Zanthoxylum | Zanthoxylum | OLON | ISOQUINOLINE |
| gillettii | Macrophyllum | African Satinwood | ALKALOID |
| | Fabara | | QUINOLINE ALKALOID |
| | macrophylla | | STEROID |
| | | | ARNOTTIANAMIDE |
| | | | AZULENE, OXYGENATED |
| | | | DERIVATIVE SESQUITERPENE |
| | | | FAGARAMIDE |
| | | | LUPEOL, TRITERPENE |
| | | | NITIDINE, 5-6-DIHYDRO: 6- |
| | | | METHOXY |
| | | | NITIDINE, 6-OXY |
| | | | OCTA-2-6-DIENE, 2-7- |
| | | | DIMETHYL |
| | | | PHELLANDRENE, ALPHA |
| | | | PIPERLONGUMINE |
| | | | SANSHOOL, GAMMA |
| | | | CHELERYTHRINE |
| | | | CHELERYTHRINE, DIHYDRO |
| | | | COUMARIN, 6-7-8- |
| | | | TRIMETHOXY |
| | | | NITIDINE |
| | | | ALPHA-CYPERONE |
| | | | N-ISOBUTYL DECA-2,4- |
| | | | DIENAMIDE |
| | | | SECURININE |
| Citrus | Citrus acida | CALAHUALA, CITRON, | FLAVANONE |
| aurantifolia | | DAMNI, EEPO OROMBO | MONOTERPENE |
| | | WEERE, KAGDI NIMBU, | NERAL |
| | | KAGHZI NIMBU, LEIMUS | PECTINESTERASE |
| | | LEMON, LEMU LIMA, | BERGAPTEN |
| | | LIMAOZINHO, LIMAU | LIMETTIN |
| | | TIPIS, LIME, MEXICAN | PIMPINELLIN, ISO |
| | | LIME, PICA LIME, WEST | JASMONIC ACID |
| | | INDIAN LIME, LIMON | LIMETTIN |
| | | AGRIA, LIMON DE, MA | XANTHOTOXIN |
| | | NAAA, MA AAO, MA- | |
| | | NAO, OFOFA-NTA, | |
| | | ORANGE, OROMBO | |
| | | WEWE, OSANWEWE, | |
| | | PETIT LIMON, PICA LIME, | |
| | | SIPORO, SITRON, SITWON | |
| | | SOUR, URIAUP SUPKABA | |

Unless defined otherwise, all technical and scientific terms used herein have the same meaning as commonly understood by one of ordinary skill in the art to which this invention pertains. Although any methods and materials similar or equivalent to those described herein can be used in the practice of the present invention, the preferred methods and materials are described.

The natural agents of the present invention are additive. The compositions of this invention are such that the natural agents in the composition interact with each other to produce a higher degree of anti-microbial and anti-viral activity than can be achieved by a single natural agent at the same dose.

The natural agents of the present invention are also synergistic. The compositions effect a coordinated or correlated action of two or more agents or physiologic processes, such that the combined action is greater than the sum of each acting separately. Further, the effects of some or all of the natural agents in compositions of this invention are supra additive, rather than additive.

The natural agents of the present invention are generally natural compounds, that is, compounds that are not synthetically or chemically prepared. Thus, the natural agents are obtained from leaves, seeds, bark, fruit, peel, flowers, roots, stems, and/or bulbs of plants, including trees, vegetables, fruits and herbs. In particular, the natural agents suitable for use in the synergistic compositions of this invention are preferably bark, root and fruit, as discussed below in detail.

The effective amount or effective dose is an amount of the synergistic composition to be administered to the host that treats HIV-related opportunistic infections or complications or inhibits the reduction of CD4 T cells. Suitable doses of a synergistic composition can be determined readily by various methods known to one skilled in the art, including generating an empirical dose-response curve, and other methods used in the pharmaceutical sciences.

The natural agents used in the compositions of this invention may be provided in the form of pure substances, or as concentrated plant extracts containing the natural agents in concentrations between about 5 to 30 percent. In a preferred embodiment, the compositions of this invention comprise 20 percent (10 mg/50 ml) of the dry plant extract. The amount of natural agent contained in a composition of this invention will depend in part on the desired results of the treatment, the stage of HIV, its associated opportunistic infections and complications, and/or AIDS and the health of the patient.

In certain embodiments of the invention, an improved method for extracting compounds from natural agents is provided whereby the efficacy of the anti-microbial and anti-fungal activity is improved. Useful levels of anti-microbial and anti-fungal agents can be extracted by various methods according to the invention to achieve a therapeutically effective result. Further, the shelf-life and stability of the active agents could be improved by these improved methods of extraction. This offers the advantage of allowing therapeutically effective doses of natural agents to be manufactured on a broad commercial scale, warehoused and distributed widely to developing countries. Until this invention, it was not known that natural agents could be extracted by methods that would increase the efficacy, shelf-life and/or stability of anti-microbial and anti-fungal activity.

Thus, the present invention relates to a method for improving the efficacy of the anti-microbial and anti-fungal agents by standardizing the specific combination of plant and plant parts used and by optimizing the extraction methods. In one embodiment, the invention relates to an extraction method in which aqueous solvents are used. In this embodiment, equal amounts in a range of one (1) to two (2) Kilograms of dried, chopped Zanthoxylum gillettii and Anogeissus leiocarpus biomass are added to two (2) gallons of boiling water.

The biomass or plant material has been previously ground into a range of 0.1-10 mm. The degree of comminutation of the plant material should provide sufficient particulate surface areas for the solvent to contact, but depends on the type of plant material used. The skilled person in this art will recognize that a variety of extraction methods are available in the literature, such as but not limited to, percolation, vent extraction, counter-current extraction, etc. In this extraction process according to the present invention water is brought to a boil. The amount of plant material to solvent, that is water, used in the extraction process, varies between 1:1 to 1:10 grams:milliliter basis with 1:1 to 1:3 being preferred.

Upon boiling for approximately thirty (30) minutes, three hundred (300) to five hundred (500) millilitres of freshly squeezed lime juice is added, and the resulting mixture is boiled for an additional three (3) hours. After cooling, the solid debris is strained from the liquid extract, and the strained liquid stored in glass or plastic containers in cool temperature. The product will remain potent for up to at least one (1) year after preparation.

In another embodiment using aqueous extraction techniques, seven and a half (7.5) Liters of water is added to cover equal amounts of the Zanthoxylum gillettii (1.5 Kilograms) and the Anogeissus leiocarpus (1.5 Kilograms) biomass. The mixture is heated until boiling and simmered at eighty (80) degrees Celsius for thirty (30) minutes. At this point, three hundred (300) to five hundred (500) millilitres of freshly squeezed lime juice is added, and the mixture is heated to a boil again for three (3) additional hours. The filtrate is then collected and spray-dried, freeze-dried or concentrating-dried to obtain dried powder extract of the dried plant extracts. The constituents in the extract are identified and quantitatively analyzed using conventional High Performance Liquid Chromatography (HPLC). Alternatively, the above process can be utilized by preparing each plant constituent separately.

Studies have shown that methanol extractions of Anogeissus leiocarpus provide a higher yield of the active extracts, and an easier-to-handle sample than aqueous extraction. Thus, changing the extraction solvent may provide greater amounts of the active compounds. For example, methanol extracts of certain Zanthoxylum species have been shown to result in greater antibacterial and anti-inflammatory chemical activity than aqueous extracts.

Accordingly, in a preferred embodiment, the method for extracting the anti-microbial and anti-fungal agents occurs via methanol extraction at a suitable temperature range, which can be achieved with ether and then subsequent steam distillation. Preferably, this extraction method is performed at a temperature range of sixty (60) to eighty (80) degrees Fahrenheit. In this preferred embodiment, equal amounts of the Zanthoxylum gillettii (310 g) and Anogeissus leiocarpus (310 g) are dried, and cut into small pieces. The chopped pieces are combined with 100-150 ml lime juice, then refluxed with methanol (1.5 L) for one and a half (1.5) hours for the first extraction, and one (1) hour each for the second and third extractions. The extracts are evaporated until dry under reduced pressure. The methanol extract is then suspended in water (500 ml) and extracted with ether (1.5 L×4) to obtain chloroform soluble fractions. Upon freeze drying or evaporation, 100 ml of the initial liquid extract results in about 1.20 g of the dried extract.

Other studies have demonstrated increased antimicrobial activity of Anogeissus leiocarpus when the natural compound is extracted via a phosphate-buffered saline solution. Thus, in another embodiment, 1 Kg of the powdered bark of each plant, Zanthoxylum gillettii, and Anogeissus leiocarpus, and three hundred (300) to five hundred (500) millilitres of freshly squeezed lime juice is added to approximately 10 L of phosphate-buffered saline (pH 7.2) in an appropriately large glass container. The combination is then extracted by stirring for four (4) days at four (4) degrees Celsius. The resultant suspension is then passed through a gauze filter to remove large debris and centrifuged at 3000×g for ten (10) minutes. The supernatant fraction is carefully decanted and passed through a 0.22 micron membrane filter under vacuum to remove insoluble materials. The clarified filtrate is then dialyzed in Spectrapor membranes with molecular limits of 3,500 and 6,000 to 8,000 against deionized distilled water. The dialysates and predialysates are then lyophilized and frozen until ready for dispensing. At this point the powder can be diluted with distilled water.

In another embodiment of the extraction, a small amount of the material is prepared for investigation purposes. Ten (10) grams of the powdered bark of each species is ground (0.2 mm sieve), combined with five (5) cubic centimetres of limejuice and defatted with hexane (200 ml) overnight at room temperature. The plant material is then extracted twice for thirty (30) minutes with methylene chloride (60 ml) at forty (40) degrees Celsius, then twice for thirty (30) minutes with methanol (60 ml) at sixty four (64) degrees Celsius, in a semi-automated Soxhlet extractor (Soxtec Avanti 2055 apparatus, Foss Tecator AB, Hoganas, Sweden). The filtrates are dried under vacuum and the residue stored at room temperature until testing. Tannins are removed from the crude methanolic extracts using Sephadex LH-20 exclusion chromatography. Methylene chloride and methanol extract are then performed according to standard methods.

In yet another embodiment, equal proportions of dried Z. gilletti and A. leiocarpus stem barks (1.0 g) are mixed with 250 ml of 30% methanol in water in a beaker. The mixture is boiled for 1 hour at 70 degrees Celsius and the mixture is allowed to cool. 20 to 30 ml of C. aurantifolia (lime) juice, is added and vortexed. The mixture is then boiled for another hour at 70 degrees Celsius. The mixture is centrifuged at 3500 g for five (5) minutes and the supernatant is collected. The residue is re-extracted with 100 ml of 30% methanol in water for one hour at 75 degrees Celsius. The combined 30% methanolic supernatants obtained after centrifugation are combined and the solvent evaporated off. The dried extract is then dissolved in 50 ml of water and stored in the refrigerator at 4 degrees Celsius.

Another embodiment of the present invention included improved methods for using the compounds extracted from natural agents whereby the efficacy of the anti-microbial and anti-viral activity is improved. It was discovered that various methods of administering the present invention to a host achieve therapeutically effective results, thus allowing therapeutically effective doses of natural agents to be administered to patients that suffer from various medical conditions, for example, conditions that make oral administration and digestion difficult. Until this invention, it was not known that natural agents could be administered by methods that would increase the efficacy of anti-microbial and anti-fungal activity in the host.

The present invention thus provides methods of treating HIV-related opportunistic infections and complications comprising administering a composition of this invention to a host in need of therapy. The doses, routes of administration, and carriers and/or adjuvants used may vary based on the view of known procedures for treatment of HIV and AIDS.

One feature of the method of using the invention is that the composition can be administered in the form of the plant material in a tablet, capsule or other pharmacologically appropriate carrier, in a parenteral solution, in a suppository, in the form of a tea, or in the form without plant material in a tablet, capsule or other pharmacological carrier, in a parentaral solution, or in a suppository which contains at least one group of the anti-microbial and anti-fungal agents extracted from the plant material.

Preferably, the compositions of this invention are administered as a solution; however, other oral or parenteral administration can be used. For example, a compound with poor solubility in acidic media may show poor or erratic bioavailability when absorbed orally. Further, intravenous administration requires that a drug be administered in a soluble form. Compounds that are intended for oral administration but are susceptible to rapid degradation at low pH (i.e. gastric acids) will likely require protection from low pH environments like the stomach. Protection can often be afforded by administering the drug in a dosage form with an acid-resistant coating. Thus, while it is possible to administer the compositions of this invention alone, the compositions may also be administered as part of a formulation. For oral administration, the compositions of this invention can be used in the form of tablets, capsules, granules, powders, lozenges, syrups, elixirs, solutions, suspensions, and the like, in accordance with standard pharmaceutical practice. A dried extract can be compounded into tablets, capsules, or other solid dosage form. A solubilized liquid formulation can be combined with syrup or other agent to formulate suspensions, solutions, elixirs, or tinctures to improve the taste, potency, or shelf life.

For parenteral administration, which includes intramuscular, intraperitoneal, subcutaneous and intravenous use, sterile solutions of the natural agents are usually prepared, and the pH of the solutions are suitably adjusted and buffered. For intravenous use, the total concentration of solutes should be controlled to render the preparation isotonic (e.g. in the range of 280 to 310 milliosmoles per liter).

Carriers useful in formulating the preparations are commonly used pharmaceutically acceptable non-toxic carriers such as gelatin, lactose sodium citrate, salts of phosphoric acid, starch, magnesium stearate, sodium lauryl sulfate, talc, polyethylene glycol, etc. The carrier may be used with other additives such as diluents, binders, buffer agents, preservatives, sweetening agents, flavoring agents, glazes, disintegrators, coating agents emulsifying agents, suspending agents, etc.

The dosage regimen may be regulated according to the potency of the individual natural agents utilized in the compositions of this invention, the mode of administration, and the needs of the host depending on factors such as the degree and severity of the disease state and age and general condition of the host being treated. Dosing ranges from five (5) to twenty (20) milligrams of the composition of the present invention per kilogram of body weight, two (2) to three (3) times daily, for one (1) to six (6) weeks depending upon the severity and length of HIV infection. Patients exhibiting clear symptoms of HIV progression (wasting, opportunistic infections, etc.) will take twenty (20) milligrams per kilogram, three (3) times daily, for six (6) weeks.

In one embodiment, the amounts of the anti-microbial and anti-fungal activities of this invention needed to be effective can be as low as five (5) milligrams per kilogram, ingested two (2) times daily, and the low dosage effective range is from five (5) milligrams per kilogram to ten (10) milligrams per kilogram, ingested up to two or three times daily, for one to six weeks. Still further, the preferred use of this invention is to administer doses of from five (5) to twenty (20) milligrams per kilogram, up to two or three times daily, making a total daily doses of about fifteen (15) to sixty (60) milligrams per kilogram per day for six weeks.

### SPECIFIC EXAMPLES

The following embodiments are for illustrative purposes only and are not intended nor should they be interpreted to limit the scope of the application.

### Example 1

The formulation of the present invention was tested for toxicity at the Centre for Scientific Research into Plant Medicine, Mampong, Ghana, the results of which can be found in Table 5, below. In this experiment, 30 male Swiss albino rats were divided into 5 groups. Each group of (6) rats received an intraperitoneal injection of varying dosages of the formulation (400, 526, 693, 912, or 1200 mg/kg of the formulation). The LD-50 (Lethal Dose-50) in laboratory animals was established at 757 mg/kg, which is the dose that can be expected to result in the death of 50% of the laboratory rats. This study reported no deaths in the 400 mg/kg group. At the 1,200 mg/kg dosage, symptoms of acute toxicity included crawling gait, tremors, twitching, and ultimately death. It is established that almost 97 times the normal adult dose is required to produce the effects of acute toxicity. No symptoms were observed in rats that received the recommended dosage.

TABLE 5

| | | | | | Approx. | |
|---|---|---|---|---|---|---|
| | | | | Time of | lethal dose | |
| | No. of | | | deaths & | & method | |
| Species & | animals & | Route of | Formulation | period of | of | |
| Strain | sex/group | Administration | & Dosage | observation | calculation | Symptoms |
| Swiss | 30; male; | Intraperitoneal | Herbal | Highest dose | 757 mg/kg | Crawling |
| albino | 5 groups | | decoction; | caused death | body | gait, |
| rats | (n = 6) | | 400, 526, | at 8 hours after | weight; | twitching |
| | | | 693, 912, | administration; | Probit | & tremor |
| | | | 1200 mg/kg | 24 hours | analysis | before |
| | | | | | | death |

The following clinical case study was conducted in an outpatient setting in Accra, Ghana, the patient was subsequently followed in Washington, D.C. The purpose of the study was to investigate the effectiveness of the herbal composition of the present invention in treating an HIV-infected patient.

Methods: A forty-one year old African female patient, diagnosed with HIV infection, was treated with anti-virus regimens by the herbal composition of the present invention on an outpatient basis.

Results: The patient was confirmed with HIV-infection by ELISA in earlier period. Patient reported a 4-year history of HIV infection. In August 2003, patient complained of lack of appetite, and had shown a marked weight loss of over 30 pounds in the previous six months. The patient's current weight was 135 pounds. The patient also reported having regular bouts of diarrhea. While AIDS wasting is defined as the involuntary loss of more than 10% of body weight, plus more than 30 days of either diarrhea, or weakness and fever, a presumptive diagnosis of anorexia was made. The result of a physical examination was normal, within normal limits.

In order to stimulate appetite, the patient was started on a regimen of 180 cc (six ounces—@20 mg/kg) of the herbal decoction three times daily. After several days, the patient reported increased appetite, greater stamina, improved mood and increased energy. Further, the patient denied any adverse effects. The patient was asked to continue on a regimen of 180 cc three times daily for a period of six weeks.

Conclusion: The herbal composition was effective in reducing symptoms of loss of appetite, and increase the energy of the patient.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents. (There have been no reported adverse effects in humans, though the combination is not recommended for use in pregnant women due to a lack of documentation regarding effects on the fetus.)

### Example 3

A clinical case series was conducted assessing the use of the present composition in 5 consecutive patients diagnosed with confirmed HIV infection, and/or confirmed chronic weight loss. Patients presented with weakness, rash, loss of appetite, weight loss, coughing and headache. Patients were treated for 4-16 weeks with doses ranging from five (5) to thirty (30) milligrams per kilogram, two (2) to three (3) times daily, for four (4) to six (6) weeks. No side effects were noted over the course of treatment, and each patient reported improvement in clinical symptoms. Each patient was treated in an Outpatient Clinic in Hohoe, Ghana, West Africa. The monitoring of each patient is currently ongoing. Data are presented below in Table 6.

TABLE 6

| Patient | Age | Gender | HIV Status | Symptoms | Outcome |
|---|---|---|---|---|---|
| 1 | 36 | Male | Positive | Loss of appetite, general | Improvement in |
| | | | | weakness, fever, rash, | appetite, resolution of |
| | | | | itching, headache | symptoms |
| 2 | 32 | Female | Positive | Loss of appetite, weight | Improvement in |
| | | | | loss | appetite, weight gain |
| | | | | | (at least 5% body |
| | | | | | weight) |
| 3 | 29 | Male | Negative | Loss of appetite, general | Improvement in |
| | | | | weakness, fever, rash, | appetite, resolution of |
| | | | | itching, headache | symptoms |
| 4 | 45 | Female | Negative | Loss of appetite, weight | Improvement in |
| | | | | loss, diarrhea, insomnia, | appetite, weight gain |
| | | | | headache, weakness | (at least 5% body |
| | | | | | weight) |
| 5 | 42 | Male | Negative | Loss of appetite, weight | Improvement in |
| | | | | loss, fever, weakness, | appetite, weight gain |
| | | | | rigors | (at least 5% body |
| | | | | | weight) |

### Example 4

ICBG plant samples were tested using an HIV-1 cytoprotection assay. This assay is based on the premise that HIV-1_{RF} is cytopathic and kills CEM-SS cells as it replicates in the culture. Compounds that inhibit HIV-1 replication are therefore able to reduce the amount of viral cytopathic effect that occurs in the culture. As a result, compound activity is measured by determining the amount of cell viability remaining in the cultures following the six day incubation period. The % reduction in viral cytopathic effect is a direct measure of compound activity. The greater the % reduction in viral cytopathic effect, the greater the antiviral activity of the compound.

Similarly, compound cytotoxicity is measured as the % reduction in cell viability resulting from incubation of the compound with the cells (in the absence of virus). The greater the reduction in cell viability, the greater the cytotoxicity of the compound.

The results, summarized below in Table 7, for the testing of ICBG plant samples indicate no specific antiviral activity against HIV-1_{RF} was detected for samples SU-3100 and SU-3102. Furthermore, SU-3100 was toxic at a concentration of 200 μg/ml and SU-3102 was toxic at concentrations of 200 and 62.5 μg/ml.

In contrast, compound SU-3101 (the present invention) exhibited moderate antiviral activity in the assay with 100% reduction in viral cytopathic effects at 62.5 μg/ml and an IC50 value of 29.8 μg/ml. SU-3101 was toxic at the high-test concentration of 200 μg/ml, resulting in a TC50 value of 131 μg/ml and an Antiviral Index of 4.4. Visual observations of the cell cultures treated with 62.5 μg/ml SU-3101 (the present invention) indicated a possible enhanced rate of cell growth (larger cell pellets observed; increased amounts of MTS staining also observed), suggesting this compound may stimulate cell replication.

The overall assay performance was judged as valid based upon the MOI-sensitive positive control compound (AZT), exhibiting the expected level of antiviral activity in the assay. Macroscopic observation of the cells in each well of the microtiter plate confirmed the cytotoxicity results obtained following staining of the cells with the MTS metabolic dye.

TABLE 7

| Compound | Assay Date | IC₅₀ | TC₅₀ | AI | Comments |
|---|---|---|---|---|---|
| SU-3100 | Aug. 4, 2004 | N/A | 132 μg/ml | N/A | No activity observed at |
| | | | | | concentrations tested; toxic at |
| | | | | | 200 μg/ml. |
| SU-3101 | Aug. 4, 2004 | 29.8 μg/ml | 131 μg/ml | 4.4 | Moderate antiviral activity; |
| (the present | | | | | toxic at 200 μg/ml. |
| invention) | | | | | |
| SU-3102 | Aug. 4, 2004 | N/A | 41.3 μg/ml | N/A | No activity observed at |
| | | | | | concentrations tested; toxic at |
| | | | | | 200 and 62.5 μg/ml. |
| AZT | Aug. 4, 2004 | <0.002 μM | >0.5 μM | >320 | Positive Control Compound |

The accompanying drawings, which are included to provide a further understanding of the invention and are incorporated in and constitute a part of this specification, illustrate embodiments of the invention and together with the description serve to explain the principles of the invention. Formula 1 depicts the chemical structure of Chelerythrine, the chemical believed to be one of the more active compounds in the extract of Zanthoxylum gilletii.

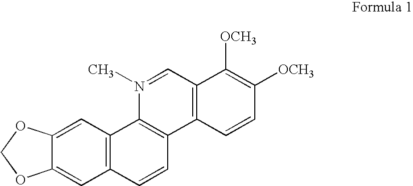

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A composition comprising Zanthoxylum gillettii, Anogeissus leiocarpus, and citrus juice.

2. A composition comprising biomass extracts isolated from Zanthoxylum gillettii, Anogeissus leiocarpus, and citrus juice.

3. The composition of claim 1, wherein said citrus juice comprises a lime juice.

4. The composition of claim 2, wherein said biomass extracts isolated from Anogeissus leiocarpus comprises one or more of Coumarin; Flavellagic Acid; 3-3′-4′Trimethoxy; 4′-O-Beta-D′-Glucoside; and 3-4-3′-Tri-O-Methylflavellagic Acid.

5. The composition of claim 2, wherein said biomass extracts isolated from Zanthoxylum gillettii comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine.

6. The composition of claim 2, wherein said biomass extracts isolated from citrus juice comprises one or more of Flavanone; Monoterpene; Neral; Pectinesterase; Bergapten; Limettin; Pimpinellin,ISO; Jasmonic Acid; and Xanthotoxin.

7. The composition of claim 1, further comprising a pharmaceutically acceptable carrier, excipient or dilutant.

8. The composition of claim 6, in the form of a capsule, tablet, liquid or powder.

9. The composition of claim 1 wherein said Zanthoxylum gillettii comprises one or more of Olon and African Satinwood, and said Anogeissus leiocarpus comprises one or more of Atara, Ayin, Kassanki, Kedeli, Kerekete, Kerkete, Kodyoli, Marike, Marke, N′galama, Ngalama, Pako Ayin, Pako Dudu, Sahab, and said citrus juice comprises one or more of Calahula, Citron, Damni, Eepo Orombo Weere, Kagdi Nimbu, Kaghzi Nimbu, Leimus Lemon, Lemu Lima, Limaozinho, Limau Tipis, Lime, Mexican Lime, Pica Lime, West Indian Lime, Limon Agria, Limon De, Ma Naaa, Ma Aao, Ma Nao, Ofofa-Nta, Orange, Orombo Wewe, Osanwewe, Petit Limon, Pica Lime, Siporo, Sitron, Sitwon Sour, Uriaup Supkaba.

10. The composition of claim 2, wherein said Zanthoxylum gillettii is in the form of concentrated plant extracts containing one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine and said Anogeissus leiocarpus is in the form of concentrated plant extracts containing one or more of Coumarin; Flavellagic Acid; 3-3′-4′Trimethoxy; 4′-O-Beta-D′-Glucoside; and 3-4-3′-Tri-O-Methylflavellagic Acid in concentrations between 5 to 30% by weight.

11. The composition of claim 2, wherein said Zanthoxylum gillettii is in the form of concentrated plant extracts containing one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine and said Anogeissus leiocarpus is in the form of concentrated plant extracts containing one or more of Coumarin; Flavellagic Acid; 3-3′-4′Trimethoxy; 4′-O-Beta-D′-Glucoside; and 3-4-3′-Tri-O-Methylflavellagic Acid in concentrations of 20% (10 mg/50 mL) by weight.

12. The composition of claim 1 in the form of a liquid wherein the concentration of Zanthoxylum gillettii, Anogeissus leiocarpus and lime juice, or biomass extracts isolated therefrom, is in the range from 20 μg/ml to 300 μg/ml.

13. The composition of claim 1 in the form of a liquid wherein the concentration of Zanthoxylum gillettii, Anogeissus leiocarpus and lime juice, or biomass extracts isolated therefrom, is in the range from 40 μg/ml to 200 μg/ml.

14. The composition of claim 1 wherein said Zanthoxylum gillettii, Anogeissus leiocarpus and lime juice, or biomass extracts isolated therefrom are administered in a daily dose from 1 to 200 milligrams per kilogram of body weight per day.

15. The composition of claim 1 wherein said Zanthoxylum gillettii, Anogeissus leiocarpus and lime juice, or biomass extracts isolated therefrom are administered in a daily dose from 15 to 60 milligrams per kilogram of body weight per day.

16. A method for treating or preventing immunodeficiency virus related opportunistic infections or complications which comprises administering a pharmaceutically effective amount of a composition comprising Zanthoxylum gillettii, Anogeissus leiocarpus, and citrus juice to a human or mammal.

17. A method for treating or preventing immunodeficiency virus related opportunistic infections or complications which comprises administering a pharmaceutically effective amount of a composition comprising biomass extracts isolated from Zanthoxylum gillettii, Anogeissus leiocarpus, and citrus juice to a human or mammal.

18. A method for reducing viral load, increasing CD4 count and decreasing immunodeficiency virus related symptoms which comprises administering a pharmaceutically effective amount of a composition comprising Zanthoxylum gillettii, Anogeissus leiocarpus, and citrus juice to a human or mammal.

19. A method for reducing viral load and increasing CD4 count which comprises administering a composition comprising Zanthoxylum gillettii, Anogeissus leiocarpus, and citrus juice in a pharmaceutically acceptable amount to an individual in need thereof.

20. A method for decreasing immunodeficiency virus related symptoms comprises administering a composition comprising Zanthoxylum gillettii, Anogeissus leiocarpus, and citrus juice in a pharmaceutically acceptable amount to an individual in need thereof.

21. A method of treating immunodeficiency virus related opportunistic complications, reducing the depletion of CD4 T cells, decreasing viral load, diarrhea, fatigue, anorexia, cough, fever, and increasing weight, comprising delivering to said patient a composition comprising Zanthoxylum gillettii, Anogeissus leiocarpus, and citrus juice, or biomass extracts isolated therefrom, in an amount effective to reduce and/or prevent immunodeficiency virus related opportunistic complications, the depletion of CD4 T cells, viral load, diarrhea, fatigue, anorexia, cough, fever, and weight loss in an individual in need thereof.

22. The method of claim 21, wherein said composition is administered in the form of the plant material in a tablet, capsule, pharmacological carrier, parenteral solution, suppository, or liquid.

23. The method of claim 21, wherein said composition is administered in the form without the plant material in a tablet, capsule, pharmacological carrier, parenteral solution, suppository, or liquid, which contains at least one group of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid.

24. A method for isolating a mixture of biomass extracts from organic compounds comprising subjecting a starting material to liquid extraction with a liquid organic extractant in which said biomass extracts are soluble; recovering said biomass extracts from said extractant whereby said biomass extracts are isolated from said organic compounds contained in said starting material and said isolated biomass extract comprises at least one of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

25. The method of claim 24 wherein said liquid organic extractant is selected from the group comprising: water, acetone, toluene, benzene, ethanol, heptane, hexane, pentanone, methanol, propanol, isopropanol, ethyl acetate, diethyl ether, trichloroethane, methyl ethyl ketone, n-butanol, 1,2-dichloroethane, dichloromethane, chloroform and mixtures thereof; and the ratio of starting material to solvent used in the extraction process is between 1:1 to 1:10 grams:milliliter.

26. The method of claim 24 wherein said starting material comprises one or more of Zanthoxylum gillettii, Anogeissus leiocarpus and citrus.

27. The method of claim 24 further comprising adding citrus to said isolated biomass extract and boiling the mixture for 1 to 6 hours.

28. A method for isolating a mixture of biomass extracts from organic compounds comprising subjecting a starting material to liquid extraction with a liquid organic extractant in which said biomass extracts are soluble; recovering said biomass extracts from said extractant whereby said biomass extracts are isolated from said organic compounds contained in said starting material and said isolated biomass extract comprises at least one of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine.

29. The method of claim 28 wherein said starting material is Zanthoxylum gillettii.

30. A method for isolating a mixture of biomass extracts from organic compounds comprising subjecting a starting material to liquid extraction with a liquid organic extractant in which said biomass extracts are soluble; recovering said biomass extracts from said extractant whereby said biomass extracts are isolated from said organic compounds contained in said starting material and said isolated biomass extract comprises at least one of Coumarin; Flavellagic Acid; 3-3′-4′Trimethoxy; 4′-O-Beta-D′-Glucoside; and 3-4-3′-Tri-O-Methylflavellagic Acid.

31. The method of claim 30 wherein said starting material is Anogeissus leiocarpus.

32. A method for isolating a mixture of biomass extracts from organic compounds comprising subjecting a starting material to liquid extraction with a liquid organic extractant in which said biomass extracts are soluble; recovering said biomass extracts from said extractant whereby said biomass extracts are isolated from said organic compounds contained in said starting material and said isolated biomass extract comprises at least one of Flavanone; Monoterpene; Neral; Pectinesterase; Bergapten; Limettin; Pimpinellin,ISO; Jasmonic Acid; and Xanthotoxin.

33. The method of claim 30 wherein said starting material is citrus.

34. A method for isolating a mixture of biomass extracts from organic compounds comprising subjecting a starting material to water thereby producing an extractant; recovering said biomass extract from said extractant whereby said biomass extract is isolated from said organic compounds contained in said starting material and said isolated biomass extract comprises at least one of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid.

35. The method of claim 34 wherein said recovering step is achieved by reducing the temperature of said extractant to solidify said biomass extract; recovering said solidified biomass extract by spray-drying, freeze-drying or concentrating-drying to obtain dried powder biomass extract.

36. A method for isolating a mixture of biomass extracts from organic compounds comprising subjecting a starting material to water; boiling the mixture for 10 to 60 minutes, adding citrus; boiling the mixture again for 1 to 6 hours; reducing the temperature of said mixture to solidify said biomass extract; recovering said solidified biomass extract by spray-drying, freeze-drying or concentrating-drying to obtain dried powder biomass extract.

37. A method for isolating a mixture of biomass extracts from organic compounds comprising subjecting a starting material to methanol thereby producing an extractant; drying said extractant; adding said extractant to water; subjecting said extractant to ether to obtain chloroform soluble fractions; recovering said biomass extract from said extractant whereby said biomass extract is isolated from said organic compounds contained in said starting material and said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

38. The method of claim 37 wherein said starting material comprises one or more of Zanthoxylum gillettii, Anogeissus leiocarpus and citrus.

39. A method for isolating a mixture of biomass extracts from organic compounds comprising subjecting a starting material to phosphate-buffered saline thereby producing an extractant; recovering said biomass extract by filtering or centrifuging said extractant; washing said recovered biomass extract; dialyzing said recovered biomass extract; lyophilizing said recovered biomass extract; freezing said recovered biomass extract, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

40. The method of claim 39, further comprising diluting said recovered biomass extract with water.

41. The method of claim 39 wherein said starting material comprises one or more of Zanthoxylum gillettii, Anogeissus leiocarpus and citrus.

42. A method for isolating a mixture of biomass extracts from organic compounds comprising subjecting a starting material to hexane thereby producing an extractant; adding methylene chloride to said extractant; adding methane to said extractant; recovering said biomass extract by drying or subjecting said extractant to exclusion chromatography, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

43. The method of claim 42 wherein said starting material comprises one or more of Zanthoxylum gillettii, Anogeissus leiocarpus and citrus.

44. A method for isolating a mixture of biomass extracts from organic compounds comprising subjecting a starting material to methanol thereby producing an extractant; adding citrus to said extractant; boiling said extractant; recovering said biomass extract by filtering or centrifugation; subjecting said biomass extract again to methanol to produce a further extractant; recovering a further biomass extract by filtering or centrifugation; washing said further biomass extract; and drying further biomass extract, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

45. The method of claim 44 wherein said starting material comprises one or more of Zanthoxylum gillettii and Anogeissus leiocarpus.

46. A product useful for treating or preventing immunodeficiency virus related opportunistic infections or complications which comprises isolating a mixture of biomass extracts from organic compounds comprising subjecting a starting material to liquid extraction with a liquid organic extractant in which said biomass extracts are soluble; recovering said biomass extracts from said extractant whereby said biomass extracts are isolated from said organic compounds contained in said starting material and said isolated biomass extract comprises at least one of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

47. A product useful for reducing viral load and increasing CD4 count which comprises isolating a mixture of biomass extracts from organic compounds comprising subjecting a starting material to liquid extraction with a liquid organic extractant in which said biomass extracts are soluble; recovering said biomass extracts from said extractant whereby said biomass extracts are isolated from said organic compounds contained in said starting material and said isolated biomass extract comprises at least one of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

48. A product useful for treating immunodeficiency virus related opportunistic complications, reducing the depletion of CD4 T cells, decreasing viral load, diarrhea, fatigue, anorexia, cough, fever, and increasing weight, comprising isolating a mixture of biomass extracts from organic compounds comprising subjecting a starting material to liquid extraction with a liquid organic extractant in which said biomass extracts are soluble; recovering said biomass extracts from said extractant whereby said biomass extracts are isolated from said organic compounds contained in said starting material and said isolated biomass extract comprises at least one of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

49. A product useful for treating or preventing immunodeficiency virus related opportunistic infections or complications which comprises isolating a mixture of biomass extracts from organic compounds comprising subjecting a starting material to methanol thereby producing an extractant; drying said extractant; adding said extractant to water; subjecting said extractant to ether to obtain chloroform soluble fractions; recovering said biomass extract from said extractant whereby said biomass extract is isolated from said organic compounds contained in said starting material and said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

50. A product useful for reducing viral load and increasing CD4 count which comprises isolating a mixture of biomass extracts from organic compounds comprising subjecting a starting material to methanol thereby producing an extractant; drying said extractant; adding said extractant to water; subjecting said extractant to ether to obtain chloroform soluble fractions; recovering said biomass extract from said extractant whereby said biomass extract is isolated from said organic compounds contained in said starting material and said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

51. A product useful for treating immunodeficiency virus related opportunistic complications, reducing the depletion of CD4 T cells, decreasing viral load, diarrhea, fatigue, anorexia, cough, fever, and increasing weight, comprising isolating a mixture of biomass extracts from organic compounds comprising subjecting a starting material to methanol thereby producing an extractant; drying said extractant; adding said extractant to water; subjecting said extractant to ether to obtain chloroform soluble fractions; recovering said biomass extract from said extractant whereby said biomass extract is isolated from said organic compounds contained in said starting material and said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

52. A product useful for treating or preventing immunodeficiency virus related opportunistic infections or complications which comprises subjecting a starting material to phosphate-buffered saline thereby producing an extractant; recovering said biomass extract by filtering or centrifuging said extractant; washing said recovered biomass extract; dialyzing said recovered biomass extract; lyophilizing said recovered biomass extract; freezing said recovered biomass extract, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

53. A product useful for reducing viral load and increasing CD4 count which comprises subjecting a starting material to phosphate-buffered saline thereby producing an extractant; recovering said biomass extract by filtering or centrifuging said extractant; washing said recovered biomass extract; dialyzing said recovered biomass extract; lyophilizing said recovered biomass extract; freezing said recovered biomass extract, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

54. A product useful for treating immunodeficiency virus related opportunistic complications, reducing the depletion of CD4 T cells, decreasing viral load, diarrhea, fatigue, anorexia, cough, fever, and increasing weight, comprising subjecting a starting material to phosphate-buffered saline thereby producing an extractant; recovering said biomass extract by filtering or centrifuging said extractant; washing said recovered biomass extract; dialyzing said recovered biomass extract; lyophilizing said recovered biomass extract; freezing said recovered biomass extract, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

55. A product useful for treating or preventing immunodeficiency virus related opportunistic infections or complications which comprises subjecting a starting material to hexane thereby producing an extractant; adding methylene chloride to said extractant; adding methane to said extractant; recovering said biomass extract by drying or subjecting said extractant to exclusion chromatography, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

56. A product useful for reducing viral load and increasing CD4 count which comprises subjecting a starting material to hexane thereby producing an extractant; adding methylene chloride to said extractant; adding methane to said extractant; recovering said biomass extract by drying or subjecting said extractant to exclusion chromatography, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

57. A product useful for treating immunodeficiency virus related opportunistic complications, reducing the depletion of CD4 T cells, decreasing viral load, diarrhea, fatigue, anorexia, cough, fever, and increasing weight, comprising subjecting a starting material to hexane thereby producing an extractant; adding methylene chloride to said extractant; adding methane to said extractant; recovering said biomass extract by drying or subjecting said extractant to exclusion chromatography, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

58. A product useful for treating or preventing immunodeficiency virus related opportunistic infections or complications which comprises comprising subjecting a starting material to methanol thereby producing an extractant; adding citrus to said extractant; boiling said extractant; recovering said biomass extract by filtering or centrifugation; subjecting said biomass extract again to methanol to produce a further extractant; recovering a further biomass extract by filtering or centrifugation; washing said further biomass extract; and drying further biomass extract, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

59. A product useful for reducing viral load and increasing CD4 count which comprises comprising subjecting a starting material to methanol thereby producing an extractant; adding citrus to said extractant; boiling said extractant; recovering said biomass extract by filtering or centrifugation; subjecting said biomass extract again to methanol to produce a further extractant; recovering a further biomass extract by filtering or centrifugation; washing said further biomass extract; and drying further biomass extract, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.

60. A product useful for treating immunodeficiency virus related opportunistic complications, reducing the depletion of CD4 T cells, decreasing viral load, diarrhea, fatigue, anorexia, cough, fever, and increasing weight, comprising comprising subjecting a starting material to methanol thereby producing an extractant; adding citrus to said extractant; boiling said extractant; recovering said biomass extract by filtering or centrifugation; subjecting said biomass extract again to methanol to produce a further extractant; recovering a further biomass extract by filtering or centrifugation; washing said further biomass extract; and drying further biomass extract, wherein said isolated biomass extract comprises one or more of Arnottianamide; Azulene; Sesquiterpene; Fagaramide; Lupeol; Triterpene; Nitidine,5-6-Dihydro:6-Methoxy; Nitidine,6-Oxy; Octa-2-6-Diene,2-7-Dimethyl; Alpha-Phellandrene; Piperlongumine; Gamma-Sanshool; Chelerythrine; Chelerythrine,Dihydro; Coumarin,6-7-8-Trimethoxy; Nitidine; Alpha-Cyperone; N-Isobutyl-Deca-2,4-Dienamide; Securinine, Coumarin; Flavellagic Acid; 3-3′-4′Trimethoxy; 4′-O-Beta-D′-Glucoside; and 3-4-3′-Tri-O-Methylflavellagic Acid, Flavanone; Monoterpene; Neral; Pectinesterase; Bergapten; Limettin; Pimpinellin,ISO; Jasmonic Acid; and Xanthotoxin.
